(19) United States
(12) Patent Application Publication
(10) Pub. No.: US 2010/0292363 A1
(43) Pub. Date: Nov. 18, 2010

(54) MATERIAL FOR BUILDING UP STUMPS

(75) Inventors: Stephan Neffgen, Hamburg (DE); Karsten Hauser, Hamburg (DE)

Correspondence Address: Casimir Jones, S.C., 2275 DEMING WAY, SUITE 310, MIDDLETON, WI, 53562, US

(73) Assignee: ERNST MÜHLBAUER GMBH & CO. KG, Norderfriedrichskoog (DE)

(21) Appl. No.: 12/809,871

(22) PCT Filed: Dec. 18, 2008
(86) PCT No.: PCT/EP2008/010843

§ 371 Date: Jun. 21, 2010

(30) Foreign Application Priority Data
Dec. 21, 2007 (EP) 07024908.1

(51) Int. Cl. A61K 6/083

(52) U.S. Cl. 523/115; 977/773

(57) ABSTRACT

The invention relates to a polymerizable material for the production of a dental material for building up stumps, having the following characteristics: —the material comprises at least one polymerizable monomer, oligomer, and/or pre-polymer, —the total filler content of the material is 50 to 85% by weight, —the material comprises 5 to 25% by weight nano-filler, having a mean particle size d5o of 300 nm or less, wherein at least 50% by weight of said nano-filler has a particle size of 200 nm or less, —the material comprises 35 to 75% by weight micro-filler, having a mean particle size d₅₀ of 0.3 to 10 μm, —the ability to grind the cured material for building up stumps deviates from the ability to grind human dentin by 15% at the most. The invention further relates to a kit comprising at least two components for the production of such a polymerizable material.

The invention relates to a polymerizable material for producing a dental core buildup material

A core buildup material serves to build up a severely damaged tooth, such that the tooth that has been built up can reliably support a crown or bridge After the application of the core buildup material to the damaged tooth, the material is modeled and cured Subsequently, the restored tooth is ground such that a tooth core suitable for supporting a crown or a bridge arises Core buildup materials are disclosed, for example, in EP-A-1 790 323

It is an object of the invention to provide a polymerizable material for production of a dental core buildup material, which has good processability and which allows good further processing after it has cured to form the core buildup material

According to the invention, the polymerizable material for this purpose has the following features
the material contains at least one polymerizable monomer, oligomer and/or prepolymer,
the total filler content of the material is 50 to 85% by weight,
the material contains 5 to 25% by weight of nanofiller with a mean particle size d₅₀ of 300 nm or less, at least 50% by weight of this nanofiller having a particle size of 200 nm or less,
the material contains 35 to 75% by weight of microfiller with a mean particle size d₅₀ of 0 3 to 10 μm,
the grindability of the cured core buildup material differs by at most 15% from the grindability of human dentin

The core of the invention is the introduction of a filler combination into the material, said filler combination leading to grindability of the cured core buildup material which approximates to the grindability of human dentin

To determine the grindability, test specimens are first produced from cured core buildup material In the explanation of the production of the test specimens and of the test method for determining the grindability which follows, it is assumed that a light-curing material is used The process described hereinafter is based on a method described in the literature (Thesis by Iwer Lasson, title “Mechanische Eigenschaften verschiedener Aufbaumaterialien” [Mechanical Properties of Different Buildup Materials], University Medical Center Hamburg-Eppendorf, 2005)

Test specimens of the core buildup materials are produced in a polymer mold with a length of 40 mm, a width of 10 mm and a height of 5 mm The mold is placed onto a microscope slide After the introduction of the polymerizable material, the mold is covered with film and a second microscope slide is pressed onto the material, such that the excess is displaced and can be removed The microscope slides are fixed with a spring clip Thereafter, the test specimen is exposed from both sides for 90 s each in a light oven (HeraFlash from Kulzer) Thereafter, they are removed from the mold and stored at 37° C. in distilled water for 16 h At least 4 test specimens are produced from each material

The reference used is human dentin For this purpose, the crowns of extracted human molars are ground to rectangles with a diamond saw, which removes the enamel Thereafter, the crowns are processed with the diamond saw to give dentin blocks with a width of 10 mm and a height of 5 mm (see also pages 38-39 of the thesis). Then the dentin blocks are embedded with a core buildup material such that the human dentin can be ground The core buildup material is cured as described above

Until the measurement, all test specimens are stored in distilled water at room temperature

The grinding tests are carried out on a compressed air-borne swivel table (air-borne carriage) with holding device for the test specimen, a holder for an angle piece with the grinding wheel, and a control unit for the speed of rotation, spray water and spray air A cylindrical diamond grinding wheel with a particle size of 100 μm (ISO 836 314 012 type) from Brasseler is used The holder used for the grinding wheel is a commercial angle piece with micromotor from KaVo

In the course of performance of the grindability test, the test specimens are mounted on the air-borne carriage with the aid of the holding device For fine adjustment, the test specimen is moved a distance of 0 2 mm toward the grinding wheel with the aid of a micrometer screw Then the test specimen secured on the air-borne carriage, by means of a pulling weight of 100 g, is moved past the rigidly mounted angle piece with the grinding wheel and ground in the process The speed of the angle piece is set to 150 000 rpm and, in order to satisfy the ISO 11405 standard, the grinding wheel is cooled with spray water at a flow rate of 50 ml/min and a temperature of 23° C. To determine the grindability, a stop watch is used to measure the time that the grinding wheel needs to cover a distance of 10 mm on the test specimen surface After 30 measurements in each case, a new grinding wheel is used

The method outlined corresponds to the description on pages 42 to 44 of the thesis The sole difference is the use of a pulling weight of 100 g, since this increases the reproducibility of the results

A difference in the grindability of the cured inventive material of at most 15%, preferably at most 10%, compared to the grindability of human dentin thus means, in the sense of the claim, that the time measured, in which the grinding wheel covers a distance of 10 mm on the test specimen surface, differs by at most 15% or 10% from the corresponding time in the case of grinding of human dentin

The resin component of the inventive material comprises polymerizable monomers, oligomers and/or prepolymers, which are preferably selected from the group consisting of free-radically and cationically polymerizable monomers, oligomers and/or prepolymers Free-radically polymerizable monomers, oligomers and/or prepolymers, especially free-radically polymerizable monomers, are preferred The free-radical polymerization can be initiated by suitable initiators, especially photocuring agents and/or chemical initiator substances Chemical initiator systems can be used especially when the polymerizable material is stored as a two-component or multicomponent system and is only mixed immediately before use

The suitable monomers used are more preferably acrylates, methacrylates, acrylamides, methacrylamides, vinyl ethers, epoxides, oxetanes, spiroorthocarbonates, spiroorthoesters, bicyclic orthoesters, bicyclic monolactones, bicyclic bislactones, cyclic carbonates, cyclic acetals, allyl sulfides, vinylcyclopropanes, organic phosphates, organic phosphonates, organic phosphites or a combination of these compounds Without restricting generality, some examples include methyl (meth)acrylate, ethyl (meth)acrylate, n- or i-propyl(meth)acrylate, n-, i- or tert-butyl (meth)acrylate, hexyl (meth)acrylate, 2-ethylhexyl (meth)acrylate, cyclohexyl (meth)acrylate, isobornyl (meth)acrylate, hydroxyethyl (meth)acrylate, hydroxypropyl (meth)acrylate, phosphoric esters of hydroxyethyl (meth)acrylate or hydroxypropyl (meth)acrylate, (meth)acrylic acid, malonic acid mono(meth)acrylate ester, succinic acid mono(meth)acrylate ester, maleic acid mono(meth)acrylate ester, glyceryl (meth)acrylate, glyceryl (meth)acrylate ester, glyceryl di(meth)acrylate, glyceryl di(meth)acrylate ester (for example glyceryl di(meth)acrylate succinate), 4-(meth)acryloyloxyethyltrimellitic acid, bis-4,6- or bis-2,5-(meth)acryloyloxyethyltrimellitic acid, 2-(((alkylamino)carbonyl)oxy)ethyl (meth)acrylate, allyl (meth)acrylate, butanediol di(meth)acrylate, hexanediol di(meth)acrylate, decanediol di(meth)acrylate, dodecanediol di(meth)acrylate, ethylene glycol di(meth)acrylate, diethylene glycol di(meth)acrylate, triethylene glycol di(meth)acrylate, tetraethylene glycol di(meth)acrylate, polyethylene glycol di(meth)acrylates, glyceryl di(meth)acrylate, glycerylpropoxy tri(meth)acrylate, trimethylolpropane tri(meth)acrylate, ethoxylated and/or propoxylated trimethylolpropane tri(meth)acrylates, pentaerythritol tetra(meth)acrylate, dipentaerythritol hexa(meth)acrylate, bisphenol A di(meth)acrylate, ethoxylated and/or propoxylated bisphenol A di(meth)acrylates, 2,2-bis-4-(3-(meth)acryloyloxy-2-hydroxypropoxy)phenylpropane and compounds derived therefrom, chloro- and bromophosphoric esters of bisphenol A glycidyl (meth)acrylate, urethane (meth)acrylates (for example 7, 7,9-trimethylol-4,13-dioxo-3,14-dioxa-5,12-diazahexadecane 1,16-dioxydimethacrylate), polyesterurethane (meth)acrylates, polyester (meth)acrylates, polycarbonate (meth)acrylates, polyamide (meth)acrylates, polyimide (meth)acrylates, phosphazene (meth)acrylates and siloxane (meth)acrylates; ethyl vinyl ether, n- or i-propyl vinyl ether, n-, i- or tert-butyl vinyl ether, hexyl vinyl ether, octyl vinyl ether, cyclohexyl vinyl ether, cyclohexyl 3,4-epoxy-1-methylvinyl ether, dimethanol cyclohexyl monovinyl ether, 1,4-dimethanolcyclohexyl divinyl ether, propanediol divinyl ether, butanediol divinyl ether, hexanediol divinyl ether, octanediol divinyl ether, decanediol vinyl ether, ethylene glycol divinyl ether, diethylene glycol divinyl ether, triethylene glycol divinyl ether, triethylene glycol monovinyl ether mono(meth)acrylate, polyethylene glycol divinyl ether, tripropylene glycol divinyl ether, glyceryl trivinyl ether, pentaerythritol tetravinyl ether, 7,7,9-trimethyl-4,13-dioxo-3,14-dioxa-5,12-diazahexadecane-1,16-dioxydivinyl ether, bisphenol A divinyl ether, ethoxylated and/or propoxylated bisphenol A divinyl ether, polyester vinyl ether, polycarbonate vinyl ether, polyacrylate vinyl ether, polyamide vinyl ether, polyimide vinyl ethers, polyurethane vinyl ether, phosphazene vinyl ether and siloxane vinyl ether, alkyl glycidyl ether, glycidol, glycidyl (meth)acrylate, dipentene dioxide, 1,2-epoxy-hexadecane, bis(3,4-epoxycyclohexyl) adipate, vinylcyclohexene oxides, vinylcyclohexene dioxides, epoxycyclohexane carboxylates (for example 3,4-epoxycyclohexylmethyl 3,4-epoxycyclohexenecarboxylate), butanediol diglycidyl ether, hexanediol diglycidyl ether, dodecanediol diglycidyl ether, diglycidyl ethers of the polyethylene glycols and of the polypropylene glycols, diglycidyl ethers of substituted (e g halogenated) and unsubstituted bisphenols (for example bisphenol A, bisphenol C and bisphenol F), resorcinol diglycidyl ether, trimethylolethane triglycidyl ether, trimethylolpropane triglycidyl ether, polybutadiene polyepoxides, polyester epoxides, polycarbonate epoxides, polyacrylate epoxides, polyamide epoxides, polyimide epoxides, polyurethane epoxides, phosphazene epoxides and siloxane epoxides, 3,3-disubstituted oxetanes and dioxetanes (for example 3-ethyl-3-(2-hydroxyethyl)oxetane), (trans/trans)-2,3,8,9-di(tetramethylene)-1,5,7,11-tetraoxaspira[5 5]undecane, substituted 1,3-dioxolanes (for example 2-phenyl-4-methylene-1,3-dioxolane), difunctional 6-methylene-1,4-dithiepanes, and the reaction products of nucleophilic (meth)acrylates, for example 2-hydroxyethyl (meth)acrylate or glyceryl (meth)acrylates with reactive phosphoric acid, phosphonic acid or phosphoric acid derivatives, for example P₂O₅, POCl₃ or PCl₃

Acrylates and/or methacrylates are particularly preferred polymerizable monomers Particular preference is given to bisphenol A glycidyl methacrylate (bis-GMA), triethylene glycol dimethacrylate (TEDMA), urethane dimethacrylate (UDMA), dodecane dimethacrylate, hexanediol dimethacrylate and comparable acrylates or methacrylates which are customary for dental materials

In the context of the invention, preferred lower limits for the total filler content are 55, 58 and 60% by weight Preferred upper limits are 80, 75, 72 and 70% by weight In the context of the invention, these upper and lower limits can be combined as desired to give ranges

Preferred lower limits for the nanofiller content are 6 and 8% by weight Preferred upper limits are 20 and 18% by weight The upper and lower limits mentioned can be combined as desired to give inventive ranges

The mean particle size d₅₀ of the nanofiller used in accordance with the invention is 300 nm or less, at least 50% by weight of this nanofiller having a particle size of 200 nm or less

The nanoparticles used in accordance with the invention can be used in the form of isolated particles or else agglomerated and/or aggregated particles Isolated nanoparticles are discrete structures which do not reveal any further subunits by, for example, electron microscopy methods The term “aggregated” describes highly associated structures in which the subunits detectable by electron microscopy are held together by very strong forces up to and including sinter bridges Aggregated particles generally form from initially isolated particles in secondary processes, the particles constituting the later subunits as a result of the secondary processes The term “agglomerated” describes the weak association of particles, for example owing to polar interactions between the particles which form the agglomerate Here too, the individual particles of the agglomerate are identifiable by electron microscopy

The subunits (particles) which build up aggregates and agglomerates in the context of this invention are referred to as primary particles The diameter of the primary particles can preferably be determined by transmission electron microscopy and is referred to as the primary particle size By statistically evaluating the electron micrographs of many associated primary particles, it is possible to determine means and other statistical parameters

When the nanoparticles used in accordance with the invention are dispersed in a liquid phase, they may be present in isolated form or else as agglomerates and/or aggregates according to the form used, dispersion process and surface properties of the particles The diameters of the structures (isolated particles, agglomerates, aggregates) in dispersed form are referred to as particle size The determination of the particle size can be undertaken by the analysis of the dispersions with the aid of photocorrelation spectroscopy (also known as dynamic light scattering), or else modified forms of Fraunhofer diffraction, for example PIDS technology (e g from Beckman Coulter, Europark Fichtenhain B13, 47807 Krefeld, Germany)

The mean primary particle size of the inventive nanoscale filler is preferably between 1 nm and 80 nm, more preferably between 4 nm and 60 nm and especially preferably between 6 nm and 50 nm The inventive nanofiller preferably has a BET surface area (to DIN 66131 or DIN ISO 9277) between 15 m²/g and 600 m²/g, preferably between 30 m²/g and 500 m²/g and more preferably between 50 m²/g and 400 m²/g

The nanofillers used in accordance with the invention are preferably metal, semimetal or mixed metal oxides, silicates, nitrides, sulfates, titanates, zirconates, stannates, tungstates, or a mixture of these compounds The group of the semimetals, the properties of which (in particular appearance and electrical conductivity) are between those of the metals and of the nonmetals, include boron, silicon, germanium, arsenic, antimony, bismuth, selenium, tellurium and polonium (cf Rompp Chemie Lexikon, Georg Thieme Verlag, 1990, p 1711)

The group of the metals can be found to the left of the group of the semimetals in the periodic table, i e they include the main group metals, transition group metals, lanthanides and actinides And the term mixed metal oxide, nitride, etc is understood here to mean a chemical bond in which at least two metals and/or semimetals are bonded chemically to one another together with the appropriate nonmetal anion (oxide, nitride, etc)

The nanofillers used in accordance with the invention are more preferably silicon dioxide, aluminum oxide, zirconium dioxide, titanium dioxide, zinc oxide, tin dioxide, cerium oxide, aluminum silicon oxides, silicon zinc oxides, silicon zirconium oxides, iron oxides and mixtures thereof with silicon dioxide, indium oxide and mixtures thereof with silicon dioxide and/or tin dioxide, boron nitride, strontium sulfate, barium sulfate, strontium titanate, barium titanate, sodium zirconate, potassium zirconate, magnesium zirconate, calcium zirconate, strontium zirconate, barium zirconate, sodium tungstate, potassium tungstate, magnesium tungstate, calcium tungstate, strontium tungstate and/or barium tungstate Mixed oxides of the aforementioned substances are likewise preferred

Preferred nanofillers are particles producible from fumed silicas (obtainable, for example, from Degussa under the Aerosil® trade name) according to the disclosure of WO 2005/084611 A1 Likewise preferred are nanoparticles producible from silica sols, the surface of which is organically modified, as disclosed, for example, in U.S. Pat. No. 6,899,948, EP 1 236 765 A1 and EP 803 240 A1

In the context of the invention, preferred lower limits for the microfiller content are 40 and 45% by weight Preferred upper limits are 70, 65 and 60% by weight These upper and lower limits can be combined as desired to give inventive ranges

The mean particle size (d₅₀) of the microfiller is preferably at least 0 4 μm, further preferred lower limits being 0 5, 0 7 and 1 0 μm Preferred upper limits for the mean particle size of the microfiller are 7 0, 5 0, 4 0 and 3 0 μm The upper and lower limits can be combined as desired to give inventive ranges

The microfiller may be a nonreactive filler, a reactive filler or a mixture of these two filler types A reactive filler is understood here to mean a filler which releases ions on ingress of water and can thus lead to curing of the material via an acid-base reaction These reactive fillers are used, for example, to produce compomers and glass ionomer cements and are described, for example, in D C Smith, Biomaterials 19, p 467-478 (1998)

The particle size of nano- and microfillers can be measured in different ways All of the known methods lead to absolute and hence mutually comparable determinations of the particle size Examples include static or dynamic light scattering, sedimentations in a gravitational or centrifugal field (for example ultracentrifugation), particle counting in an electrolyte (Coulter counter method), screening, image analysis or video image analysis of optical or electron microscopy images (for example transmission electron microscopy, TEM), ultrasound spectroscopy and 3D-ORM laser back-reflection Preferred methods of determining the particle size of the nano- and microfillers are specified below in connection with the examples

The microfillers used are preferably quartz powder, glass powder, glass ceramic powder, metal oxides, metal hydroxides, spherical fillers as described, for example, in DE-C 3247800, amorphous cluster fillers as described, for example, in WO 01/30306, or a mixture of these fillers

The microfillers used are more preferably barium silicate glasses, strontium silicate glasses, boroaluminosilicate glasses, phosphoaluminosilicate glasses, fluoroaluminosilicate glasses, calcium silicates, zirconium silicates, sodium aluminum silicates, sheet silicates, bentonites, zeolites including the molecular sieves, the oxides and the hydroxides of the alkali metals and alkaline earth metals, apatite, spherical fillers as described, for example, in DE-C 3247800, amorphous cluster fillers as described, for example, in WO 01/30306, or a mixture of these fillers

A preferred variant is the use of a nano- and/or microfiller in which, by organic modification, functional groups are applied to the surface of the filler, which can react chemically with the organic binder or organic resin or have a high affinity for the organic binder These functional groups are preferably acrylate, methacrylate, cyanoacrylate, acrylamide, methacrylamide, vinyl, allyl, epoxide, oxetane, vinyl ether, amino, acid, acid ester, acid chloride, phosphate, phosphonate, phosphite, thiol, alcohol and/or isocyanate groups These groups are preferably introduced via suitable compounds, for example siloxanes, chlorosilanes, silazanes, titanates, zirconates, tungstates, organic acids, organic acid chlorides or acid anhydrides

The surface of the nanofillers or microfillers is preferably organically modified by treatment with a siloxane, chlorosilane, silazane, titanate, zirconate, tungstate, or with an organic acid (as described, for example, in U.S. Pat. No. 6,387,981), an organic acid chloride or acid anhydride The siloxanes, chlorosilanes, silazanes, titanates, zirconates and tungstates more preferably have the general formulae Si(OR′)ₙR₄₋ₙ, SiClₙR₄₋ₙ, (RₘR″₃₋ₘSi)₂NH, Ti(OR′)ₙR₄₋ₙ, Zr(OR′)ₙR₄₋ₙ and W(OR′)ₙR₆₋ₙ, where m and n are each 1, 2 or 3, preferably, n=3 The R′ group bonded via oxygen, and likewise R″, is any organic functional group, preferably an alkyl group and more preferably a methyl, ethyl, propyl or isopropyl group The functional R group is any organic group and is bonded directly via a carbon atom to the silicon, titanium, zirconium or tungsten When m or n is 1 or 2, the R groups may be the same or different R is preferably selected such that it possesses one or more functional groups which can react chemically with the organic resin or have a high affinity for the organic resin These functional groups are also present in the organic acids, acid chlorides and acid anhydrides listed above, which are likewise usable for organic surface modification They are preferably acrylate, methacrylate, cyanoacrylate, acrylamide, methacrylamide, vinyl, allyl, epoxide, oxetane, vinyl ether, amino, acid, acid ester, acid chloride, phosphate, phosphonate, phosphite, thiol, alcohol and/or isocyanate groups The by-products formed in the organic surface modification of the fillers, for example alcohols, hydrochloric acid or ammonia, are preferably removed in the further steps down to possible residues (impurities), i e they are present in the dental material produced later only in small amounts of ≦0 4% by weight, preferably ≦0 2% by weight, if at all

As known in the prior art, microfillers can be surface-modified in dry form or in suspension With regard to silanization and silanization processes, reference is made to Ralf Janda, Kunststoffverbundsysteme [Polymer composite systems], 1st edition, VCH-Verlagsgesellschaft Weinheim, 1990, p 93 ff, and the literature cited there

The solvent in which the organic surface modification of the nanofillers is carried out is preferably a polar aprotic solvent and more preferably acetone, butanone, ethyl acetate, methyl isobutyl ketone, tetrahydrofurane or diisopropyl ether In addition, the direct organic surface modification of the nanofillers in the organic binder to be used to produce the dental materials is a particularly preferred procedure In this case, the organic resin or binder is the solvent to be used To accelerate the organic surface modification of the nanofillers, an acid can be added as a catalyst In each case, catalytic amounts of water, preferably between 0 01% and 5%, must be present in order to carry out the modification This water is often already present as adsorbate on the surfaces of the fillers used as the starting substance To promote the reaction, further water can be added, for example also in the form of a dilute acid

In order to accelerate the decomposition of any agglomerates and aggregates of nanofillers in the organic surface modification in the organic solvent, an additional energy input by the customary methods can be effected before or during the modification This can be accomplished, for example, by means of a high-speed stirrer, a dissolver, a bead mill or a mixer In the case of use of relatively high-viscosity solvents, this is the preferred procedure, i e particularly when the organic binder is used directly as the solvent When the organic resin or binder is not used as the solvent, the organic binder to be used can be filled directly with the dispersion of the organically modified nanofiller in the organic solvent In this case, the solvent is drawn off after the preparation of the mixture from organic binder and organically modified nanofiller The organically modified nanofiller is preferably freed of the solvent and processed further as a dry powder In this case, the dry organically modified nanopowder is then added to the organic binder and incorporated with mechanical energy input The incorporation can be effected, for example, by means of a high-speed stirrer, a dissolver, a bead mill, a roll mill, a kneader or a mixer

In the case of use of relatively high-viscosity solvents and especially in the case of direct use of the organic binder as a solvent, it may be the case that any excesses and/or unconverted portions of the compound used for organic surface modification of the nanofillers cannot be removed from the dispersion In this case, it is the preferred procedure that these possible excesses and/or unconverted portions of the compound used for organic surface modification of the nanofillers are converted by reaction with a suitable agent to substances which are then either removed from the dispersion or else can remain in the dispersion if they are safe to humans, i e are not harmful A particularly preferred procedure is the use of water as an agent, which is reacted with any excesses and/or unconverted portions of the compound used for organic surface modification of the nanofillers

A particularly preferred variant is the use of a microfiller which is radiopaque and is incorporated into the inventive material in such amounts that the inventive material has a radiopacity (to ISO 4049-2000) of preferably ≧100% Al and more preferably 200% Al

The inventive polymerizable material may be configured as a storable one-component system, which is cured, for example, by means of a photoinitiator and light In the context of the invention, however, preference is given to configuration as a multicomponent system, especially two-component system Accordingly, the invention also provides a kit for producing an inventive polymerizable material, said kit comprising at least two components from which the polymerizable material can be made up

In an inventive kit, the fillers may be present in both components Alternatively, it is possible to introduce the nanofiller only into one component of the kit, the microfiller is preferably present in both components of the kit

Such a two-component kit can be mixed either by hand or automatically In the case of automatic mixing, the two components are stored in a double-chamber cartridge and mixed homogeneously with one another by pressing out through a mixing cannula attached to this cartridge The components can be pressed out either manually or with the aid of a suitable discharge unit, which generally allows a higher expulsion force Automatic mixing is preferred

In order to ensure this automatic miscibility, it is preferred in the context of the invention that the dynamic viscosity of each component at a shear stress of 1500 Pa is between 5 and 250 Pas The dynamic viscosity is measured by the process described below in connection with the examples

Preferred lower limits for the dynamic viscosity at a shear stress of 1500 Pa are 8 and 10 Pas Preferred upper limits are 225, 200 and 175 Pas These lower and upper limits can be combined as desired to give inventive ranges

To adjust particular properties, the inventive material may additionally comprise further constituents, also including so-called additives or modifiers Without restricting generality, some examples are as follows filled and/or unfilled chip polymers, filled and/or unfilled bead polymers These filler constituents are covered by the term “microfiller” used in claim 1 and are included in the total filler content when the mean particle size thereof is within the ranges defined for microfillers When the mean particle size is above this range, these fillers are part of the additional constituents and not of the total filler content Such additional constituents may also be fumed silicas, the mean particle size of which, owing to the agglomeration and/or aggregation of primary particles, is above the upper limit defined for microfillers Further possible additional constituents are inorganic and/or organic color pigments or dyes, stabilizers (for example substituted and unsubstituted hydroxyaromatics, Tinuvins, terpinenes, phenothiazine, so-called HALS (Hindered Amine Light Stabilizers) and/or heavy metal scavengers such as EDTA), plasticizers (for example polyethylene glycols, polypropylene glycols, unsaturated polyesters, phthalates, adipates, sebacates, phosphoric esters, phosphonic esters and/or citric esters), ion-releasing substances, especially those which release fluoride ions (for example sodium fluoride, potassium fluoride and/or quaternary ammonium fluorides), radiocontrast agents (for example yttrium fluoride, ytterbium fluoride, bismuth oxide chloride and bismuth trifluoride) and/or bactericidal or antibiotic substances (for example chlorhexidine, pyridinium salts, penicillins, tetracyclines, chloramphenicol, antibacterial macrolides and/or polypeptide antibiotics)

Plasticizers (unreactive organic resin constituents) are preferably present in a proportion of 8% by weight or less Further preferred upper limits for the proportion of plasticizers are 7 and 5% by weight More preferably, the inventive material cannot contain any plasticizers whatsoever

The inventive material preferably comprises a suitable initiator system It comprises one or more initiators and optionally one or more coinitiators

As already mentioned above, the inventive material is preferably curable by free-radical polymerization

In this case, initiator(s) and coinitiator(s) may be present together in one component and/or separately in two or more components The inventive material can thus be cured chemically and/or photochemically, i e by irradiation with UV and/or visible light Dual-curing materials are preferred

The initiators usable here may, for example, be photoinitiators These are characterized in that they can bring about the curing of the material by absorbing light in the wavelength range from 300 nm to 700 nm, preferably from 350 nm to 600 nm and more preferably from 380 nm to 500 nm, and optionally by the additional reaction with one or more coinitiators Preference is given here to using phosphine oxides, benzoin ethers, benzyl ketals, acetophenones, benzophenones, thioxanthones, bisimidazoles, metallocenes, fluorones, α-dicarbonyl compounds, aryldiazonium salts, arylsulfonium salts, aryliodonium salts, ferrocenium salts, phenylphosphonium salts or a mixture of these compounds

Particular preference is given to using diphenyl-2,4,6-trimethylbenzoylphosphine oxide, benzoin, benzoin alkyl ethers, benzyl dialkyl ketals, α-hydroxyacetophenone, α-aminoacetophenone, 1-propylthioxanthone, camphorquinone, phenylpropanedione, 5,7-diiodo-3-butoxy-6-fluorone, (eta-6-cumene)(eta-5-cyclopentadienyl)iron hexafluorophosphate, (eta-6-cumene)(eta-5-cyclopentadienyl)iron tetrafluoroborate, (eta-6-cumene)(eta-5-cyclopentadienyl)iron hexafluoroantimonate, substituted diaryliodonium salts, triarylsulfonium salts or a mixture of these compounds

The coinitiators used for photochemical curing are preferably tertiary amines, borates, organic phosphites, diaryliodonium compounds, thioxanthones, xanthenes, fluorenes, fluorones, α-dicarbonyl compounds, fused polyaromatics or a mixture of these compounds Particular preference is given to using N,N-dimethyl-p-toluidine, N,N-dialkylalkylanilines, N,N-dihydroxyethyl-p-toluidine, electron-deficient tertiary amines, for example 2-ethylhexyl p-(dimethylamino)benzoate, butyrylcholine triphenylbutyl borate or a mixture of these compounds

For chemical curing at room temperature or mouth temperature, a redox initiator system is generally used, which consists of one or more initiators and (a) coinitiator(s) which serve(s) as an activator For reasons of storage stability, initiator(s) and coinitiator(s) are incorporated into spatially separate parts of the inventive dental material, i e a multicomponent, preferably a two-component, material is present The initiator(s) used are preferably inorganic and/or organic peroxides, inorganic and/or organic hydroperoxides, barbituric acid derivatives, malonylsulfamides, erotic acids, Lewis or Broensted acids or compounds which release such acids, carbenium ion donors, for example methyl triflate or triethyl perchlorate, or a mixture of these compounds, and the coinitiator(s) used are preferably tertiary amines, heavy metal compounds, especially compounds of groups 8 and 9 of the periodic table (“iron and copper group”), compounds with conically bound halogens or pseudohalogens, for example quaternary ammonium halides, weak Broensted acids, for example alcohols and water, or a mixture of these compounds

It is also possible for any conceivable combination of the above-described initiators and coinitiators to be present in the inventive dental material One example thereof is that of dual-curing dental materials which contain both photoinitiators and optionally the appropriate coinitiators for photochemical curing, and initiators and appropriate coinitiators for chemical curing at room temperature or mouth temperature

The invention is described hereinafter with reference to examples First, two test methods used in the examples are explained

### 1. Measurement of Dynamic Viscosity

As already explained above, the automatic mixing of two-component systems involves expelling the components from the particular chambers of a cartridge and mixing them homogeneously with one another in a mixing cannula In order to ensure the discharge of the material from the cartridges, the dynamic viscosity under the shear stresses which exist in the course of expulsion must not be too great The components are discharged with a force and hence under a shear stress which depends on the discharge unit used In the case of use of a commercial discharge unit, shear stresses (τ) of up to about 500 to 1500 Pa may occur

A measure of the flowability of a substance is its dynamic viscosity (η), which is calculated from the quotient of shear stress (τ) to shear rate (ý) η=τ/ý [Pas]

The dynamic viscosity of the individual components of the examples cited was determined with the aid of a Dynamic Stress Rheometer (DSR) from Rheometrics

All samples were analyzed between 2 parallel plates with a diameter of 25 mm and a gap width of 0 5 mm, with a set temperature of 23° C.

In each measurement, a suitable amount of sample was applied to the lower plate mounted in a fixed manner, and then the sample was subjected to preliminary shear with a shear stress of 600 Pa for 60 s After a rest time of 30 s, the measurement was started with the “dynamic shear stress sweep” analysis program of the DSR This involved oscillation of the upper plate with a frequency of 1 Hz and measurement of the dynamic viscosity at given shear stresses rising stepwise in steps of 10 Pa (hold time in each case 5 s) from 10 Pa to 1500 Pa

### 2. Measurement of Dimensional Stability

It is advantageous when the mixed core buildup material before curing has a certain dimensional stability, i e a sufficiently high yield point A low dimensional stability can lead in particular applications (for example applications in the upper jaw) to the material being relatively free-flowing and hence making core buildup more difficult even at rest or under low shear stresses or shear To measure the dimensional stability, the test described below can be carried out

This test derives from a CRA (Clinical Research Associates) test method, with which the viscosity or the dimensional stability of sealants was assessed qualitatively (see CRA Newsletter, August 2001)

In the upper region of a glass plate, 230 mg of the automatically mixed core buildup material were applied in each case Immediately thereafter, the glass plate was held vertically for 30 s, as a result of which the materials flowed downwards at different rates Subsequently, the glass plate was placed horizontal again and the entire material strand was cured with the aid of a handheld LED lamp (Mini-LED from Acteon)

Subsequently, a slide rule was used to measure the length of the cured material strand In the context of the invention, it is preferred that the strand length is not greater than 55 mm More preferred is an upper limit of 25 mm for the strand length

### 3. Particle Size Determination of the Nanofillers

The particle size determinations were carried out by means of dynamic light scattering (3D-PCS) The method allows the determination of the proportions by weight of particle sizes in the range from 1 nm up to a few micrometers The upper limit of the method results from the fact that larger particles sediment in the test solution and thus cannot be detected

All samples were analyzed as dilute dispersions in 2-butanone with a set solids content of about 0 5% by weight This dilution was selected primarily in order to reliably rule out particle-particle interactions

### 4. Particle Size Determination of the Microfillers

The particle size distribution is detected by means of laser diffractometry (Coulter LS 130) In this process, the proportion by weight of particles which have a particular size is determined One characteristic is the d₅₀, which reports that half (50%) of the total mass of the particles has a larger size and half has a smaller size The particles are analyzed in dilute, usually aqueous dispersions

### 5. Compressive Strength

For the determination of compressive strength, 11-12 specimens of each sample were analyzed To produce the specimens, a specimen mold consisting of stainless steel, which contains a 4 mm-high (+/−0 02) and 2 mm-wide (+/−0 01) cylindrical bore, was placed on a polyethylene film which in turn was placed on a glass microscope slide The sample was introduced into the bore without bubbles, and covered again with the film and then with a further microscope slide Excess sample was pressed out and the microscope slide was fixed with a clamp The samples were exposed from both sides with a polymerization lamp (Espe Elipar II, 3m Espe AG, Germany) for 40 s The hardened specimens were sanded while wet (sanding paper, grain size P 600) and then removed from the specimen mold The specimens were stored in distilled water at 37° C. for 24 h

The compressive strength of the specimens was determined with a force meter (Zwick 2010, Zwick GmbH & Co KG, Germany) with a 10 kN load cell This used a test device consisting of two parallel plates, one of which was mounted in a fixed manner and the second was connected to the load cell The individual specimens were placed upright in the middle of the fixed plate and the second plate was then moved downward at a constant advance rate of 1 0 mm/min, such that the specimens were stressed in the direction of the longitudinal axis thereof.

### EXAMPLES 1 AND 2

These examples are two-component systems, the two components are referred to as base paste (ATL paste) and catalyst paste (K paste) The pastes have the composition (weight figures in g) according to the following table

TABLE 1

| Example 1 | | Example 2 | |
|---|---|---|---|
| ATL paste | | ATL paste | |
| Bis-GMA | 50 000 | Bis-GMA | 50 000 |
| TEDMA | 50 000 | TEDMA | 50 000 |
| Dimethyl-p- | 1 433 | Dimethyl-p- | 1 433 |
| toluidine | | toluidine | |
| Ethyl | 1 146 | Ethyl | 1 146 |
| dimethylamino- | | dimethylamino- | |
| benzoate | | benzoate | |
| Camphorquinone | 0 573 | Camphorquinone | 0 573 |
| BHT | 0 015 | BHT | 0 015 |
| Aerosil R 974 | 11 463 | Aerosil R 974 | 8 843 |
| Glass powder | 199 725 | Glass powder | 159 175 |
| silanized | | sil | |
| d50 = 2 5 μm | | d50 = 1 5 μm | |
| Nanofiller | 67 747 | Nanofiller | 23 581 |
| K paste | | | |
| (catalyst | | | |
| paste) | | K paste | |
| Bis-GMA | 50 000 | Bis-GMA | 50 000 |
| TEDMA | 50 000 | TEDMA | 50 000 |
| BPO | 0 839 | BPO | 0 839 |
| BHT | 0 226 | BHT | 0 226 |
| Methacrylic | 0 470 | Methacrylic | 0 470 |
| acid | | acid | |
| Aerosil R 974 | 11 282 | Aerosil R 974 | 8 703 |
| Glass powder | 196 563 | Glass powder | 156 653 |
| silanized | | sil | |
| d50 = 2 5 μm | | d50 = 1 5 μm | |
| Nanofiller | 66 674 | Nanofiller | 23 208 |

### Nanofiller

280 g of Aerosil® (Degussa AG) were weighed into a 4 l two-neck flask and admixed with approx 2000 g of 2-butanone The initially pasty mixture was stirred with a precision glass stirrer until a homogeneous liquid suspension formed Then 178 36 g of 3-methacryloyloxypropyltrimethoxysilane were added dropwise with a metering pump The mobile suspension was stirred at room temperature for a total of 48 hours Subsequently, the 2-butanone was slowly drawn off on a rotary evaporator The removal of the solvent left behind a white, loose, coarse-particulate porous powder, which fell apart readily

### Nanodispersion (Precursor of the Inventive Examples)

240 g of a 1 l mixture of bisphenol A diglycidyl methacrylate (bis-GMA) and triethylene glycol dimethacrylate (TEDMA) were introduced into a 1000 ml dispersing pan Then 160 g of the nanofiller were incorporated in portions of approx 20-40 g with the aid of a Dispermat (AE04-C1 from VMA-Getzmann) using a dissolver disk with a diameter of 70 mm at rotational speeds of 250 rpm to 1250 rpm within approx 60 minutes Subsequently, dispersion was continued at 2000 rpm for approx 120 minutes This formed a virtually colorless and transparent nanodispersion

### EXAMPLE 1

### A Base Paste (ATL Paste) and a Catalyst Paste (K Paste) were Prepared

### ATL Paste

1 125 g of N,N-dimethyl-p-toluidine, 0 9 g of ethyl dimethylaminobenzoate, 0 45 g of camphorquinone and 0 012 g of 3,5-bis-tert-butyl-4-hydroxytoluene (BHT) were dissolved by stirring in 132 975 g of the nanodispersion Then 9 0 g of Aerosil R974 (Degussa AG) and 156 81 g of barium silicate glass silanized with 3-methacryloyloxypropyltrimethoxysilane (GM27884 K6 from Schott AG) were incorporated homogeneously with the aid of a planetary mixer (LPV 0 3-1 from PC Laborsystem) The paste was rolled twice using a laboratory three-roll mill (50 from Exakt) (gap setting 2/2) and then devolatilized while stirring at approx 200 mbar for 30 min

### 2) K Paste

0 669 g of dibenzoyl peroxide, 0 375 g of methacrylic acid and 0 18 g of 3,5-bis-tert-butyl-4-hydroxytoluene (BHT) were dissolved by stirring in 132 975 g of the nanodispersion Then 9 0 g of Aerosil R974 (Degussa AG) and 156 81 g of barium silicate glass silanized with 3-methacryloyloxypropyltrimethoxysilane (GM27884 K6 from Schott AG) were incorporated homogeneously with the aid of a planetary mixer (LPV 0 3-1 from PC Laborsystem) The paste was rolled twice using a laboratory three-roll mill (50 from Exakt) (gap setting 2/2) and then devolatilized while stirring at approx 200 mbar for 30 min

### EXAMPLE 2

### A Base Paste (ATL Paste) and a Catalyst Paste (K Paste) were Prepared

### 1) ATL Paste

60 g of the nanodispersion were mixed homogeneously with 65 776 g of a 1 l mixture of bisphenol A diglycidyl methacrylate (bis-GMA) and triethylene glycol dimethacrylate (TEDMA) 1 458 g of N,N-dimethyl-p-toluidine, 1 167 g of ethyl dimethylaminobenzoate, 0 583 g of camphorquinone and 0 016 g of 3,5-bis-tert-butyl-4-hydroxytoluene (BHT) were dissolved in this mixture by stirring Then 9 0 g of Aerosil R974 (Degussa AG) and 162 0 g of barium silicate glass silanized with 3-methacryloyloxypropyltrimethoxysilane (GM27884 UF1 5 from Schott AG) were incorporated homogeneously with the aid of a planetary mixer (LPV 0 3-1 from PC Laborsystem) The paste was rolled twice using a laboratory three-roll mill (50 from Exakt) (gap setting 2/2) and then devolatilized while stirring at approx 200 mbar for 30 min

### K Paste

60 g of the nanodispersion were mixed homogeneously with 67 413 g of a 1 l mixture of bisphenol A diglycidyl methacrylate (bms-GMA) and triethylene glycol dimethacrylate (TEDMA) 0 867 g of dibenzoyl peroxide, 0 486 g of methacrylic acid and 0 233 g of 3,5-bis-tert-butyl-4-hydroxytoluene (BHT) were dissolved in this mixture by stirring Then 9 0 g of Aerosil R974 (Degussa AG) and 162 0 g of barium silicate glass silanized with 3-methacryloyloxypropyltrimethoxysilane (GM27884 UF1 5 from Schott AG) were incorporated homogeneously with the aid of a planetary mixer (LPV 0 3-1 from PC Laborsystem) The paste was rolled twice using a laboratory three-roll mill (50 from Exakt) (gap setting 2/2) and then devolatilized while stirring at approx 200 mbar for 30 min

### COMPARATIVE EXAMPLES 1 AND 2

As comparative examples 1 and 2, examples 2 and 3 from the prior art according to EP 1 790 323 A1 were reworked The composition thereof is described on page of this prior art in Table 1 as embodiment 2 and embodiment 3 Embodiment 2 corresponds to the present comparative example 1, embodiment 3 to the present comparative example 2

### EXAMPLE 3

### Determination of the Dynamic Viscosities

The dynamic viscosities of the components of the examples, determined at shear stresses of 500 to 1500 Pa, are compiled in Table 2 below

| | Base paste (ATL) | | | Catalyst paste (K) | | |
|---|---|---|---|---|---|---|
| | DSR viscosity η [Pas] | | | DSR viscosity η [Pas] | | |
| | τ = 500 Pa | τ = 1000 Pa | τ = 1500 Pa | τ = 500 Pa | τ = 1000 Pa | τ = 1500 Pa |
| Comp. | 38 | 46 | 51 | 85 | 105 | 106 |
| ex. 1 | | | | | | |
| Comp. | 30 | 23 | 17 | 16 | 17 | 17 |
| ex. 2 | | | | | | |
| Ex. 1 | 101 | 130 | 136 | 165 | 217 | 239 |
| Ex. 2 | 11 | 12 | 13 | 15 | 15 | 15 |

All paste combinations were deployable with a sensorily acceptable expenditure of force

### EXAMPLE 4

### Measurement of Grindability

For the examples and comparative examples, the grindability compared to human dentin is measured by the process described above Four test specimens are produced for each example or comparative example, the time reported is the mean from four measurements

All examples and comparative examples cited were transferred into black 25 ml 1 l double-chamber cartridges (part no CS 025-01-13) from Mixpac Systems AG and stored In the production of the test specimens for the measurements of grindability and compressive strength which follow, the two components were mixed automatically This was done in each case using the yellow MB 4 2-12-D mixing cannula (Mixpac Systems AG) and the DS 24-01-00 discharge unit (likewise Mixpac Systems AG)

The test results are reported in Table 3 below

TABLE 3

| | | Standard |
|---|---|---|
| | | deviations |
| | Time [s] | [s] |
| Human dentin | 9 56 | 1 48 |
| Example 1 | 9 14 | 1 46 |
| Example 2 | 9 94 | 1 43 |
| Comparative example 1 | 6 47 | 0 54 |
| Comparative example 2 | 5 89 | 1 23 |

It is evident that the grindability of the inventive examples is very similar to that of human dentin, whereas the grindability of the comparative examples of the prior art differs significantly

The grindability measurements were carried out with the above-described diamond grinding wheels from Brasseler It should be noted that other comparable diamond grinding wheels (preferably with a grain size of 100 μm) can be used for the measurements in the context of the invention Since claim 1 of the invention defines the grindability of an inventive core buildup material relative to the grindability of human dentin, it is important merely that measurement and comparative measurement are each carried out under the same conditions with the same grinding wheel

### EXAMPLE 5

### Measurement of Compressive Strength

The compressive strength of the test specimens according to the examples and comparative examples was measured by the process described above

TABLE 4

| | Compressive |
|---|---|
| | strength [MPa] |
| Example 1 | 404 ± 33 |
| Example 2 | 447 ± 26 |
| Comparative example 1 | 323 ± 18 |
| Comparative example 2 | 298 ± 20 |

It is evident that the compressive strength of the inventive core buildup materials is significantly higher than in the prior art

1. A polymerizable material for producing a dental core buildup material, with the following features:
the material contains at least one polymerizable monomer, oligomer and/or prepolymer,
the total filler content of the material is 50 to 85% by weight,
the material contains 5 to 25% by weight of nanofiller with a mean particle size d₅₀ of 300 nm or less, at least 50% by weight of this nanofiller having a particle size of 200 nm or less,
the material contains 35 to 75% by weight of microfiller with a mean particle size d₅₀ of 0.3 to 10 μm,
the grindability of the cured core buildup material differs by at most 15% from the grindability of human dentin.

2. The material as claimed in claim 1, wherein the polymerizable monomers, oligomers and/or prepolymers are selected from the group consisting of free-radically and cationically polymerizable monomers, oligomers and/or prepolymers.

3. The material as claimed in claim 2, wherein the polymerizable monomers comprise acrylates and/or methacrylates.

4. The material as claimed in claim 1, wherein the total filler content is at least 55% by weight and at most 80% by weight.

5. (canceled)

6. The material as claimed in claim 1, wherein the nanofiller content is at least 6% by weight and at most 20% by weight.

7. (canceled)

8. The material as claimed in claim 1, wherein at least 50% by weight of the nanofiller has a particle size of 150 nm or less.

9. The material as claimed in claim 1, wherein the mean primary particle size of the nanofiller is between 1 and 80 nm.

10. The material as claimed in claim 1, wherein the microfiller content is at least 40% by weight and at most 70% by weight.

11. (canceled)

12. The material as claimed in claim 1, wherein the mean particle size d₅₀ of the microfiller is at least 0.4 μm and at most 7.0 μm.

13. (canceled)

14. The material as claimed in claim 1, wherein the nanofiller and/or the microfiller is at least partly surface-modified and has, on its surface, functional groups which can react chemically with the organic matrix of the material or have a high affinity for this matrix.

15. A kit for producing a polymerizable material as claimed in claim 1, wherein the kit comprises at least two components from which the polymerizable material can be made up.

16. The kit as claimed in claim 15, wherein the nanofiller is present only in one component.

17. The kit as claimed in claim 15, the dynamic viscosity of each component at a shear stress of 1500 Pa is between 5 and 250 Pas.

18. The kit as claimed in claim 17, the dynamic viscosity of each component at a shear stress of 1500 Pa is at least 8 Pas and at most 225 Pas.

19. (canceled)

20. A dental core buildup material obtainable by allowing a polymerizable material as claimed in claim 1 to cure.

21. The use of a polymerizable material as claimed in claim 1 for producing a dental core buildup material.

22. The material as claimed in claim 1, wherein the total filler content is at least-58% and at most 75% by weight.

23. The material as claimed in claim 1, wherein the total filler content is at least 60% and at most 72% by weight.

24. The material as claimed in claim 1, wherein the nanofiller content is at least 6% and at most 18% by weight.

25. The material as claimed in claim 1, wherein at least 50% by weight of the nanofiller has a particle size of 100 nm or less.

26. The material as claimed in claim 1, wherein the mean primary particle size of the nanofiller is between 4 and 60 nm.

27. The material as claimed in claim 1, wherein the mean primary particle size of the nanofiller is between 6 and 50 nm.

28. The material as claimed in claim 1, wherein the microfiller content is at least 45% and at most 65% by weight.

29. The material as claimed in claim 1, wherein the microfiller content is at least 45% and at most 60% by weight.

30. The material as claimed in claim 1, wherein the mean particle size d₅₀ of the microfiller is at least 0.5 μm and at most 5.0 μm.

31. The material as claimed in claim 1, wherein the mean particle size d₅₀ of the microfiller is at least 0.7 μm at most 4.0 μm.

32. The material as claimed in claim 1, wherein the mean particle size d₅₀ of the microfiller is at least 1.0 μm at most 3.0 μm.

33. The kit as claimed in claim 17, wherein the dynamic viscosity of each component at a shear stress of 1500 Pa is at least 10 Pas and at most 200 Pas.

34. The kit as claimed in claim 17, wherein the dynamic viscosity of each component at a shear stress of 1500 Pa is at least 10 Pas and at most 175 Pas.
